(19) United States
(12) Patent Application Publication
(10) Pub. No.: US 2007/0103270 A1
(43) Pub. Date: May 10, 2007

(54) Input device for the activation and control functions of plural apparatuses of a dentist's or dental treatment station/work station

(75) Inventors: Hermann Gmeinder, Biberach (DE); Richard Schoenenberger, Leutkirch (DE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Kaltenbach & Voigt GmbH, Biberach (DE)

(21) Appl. No.: 10/575,413

(22) PCT Filed: Oct. 8, 2004
(86) PCT No.: PCT/EP04/11263

§ 371 Date: Nov. 6, 2006

(30) Foreign Application Priority Data
Oct. 8, 2003 (DE) 103 46 668.1

(51) Int. Cl. G05B 23/02; A61B 5/05

(52) U.S. Cl. 340/3.43; 600/407

(57) ABSTRACT

An input device for the activation and control of functions of at least one apparatus of a dentist's or dental treatment station/work station, wherein the input device has a first input element for the generation of navigation information for the control of a pointer on a user interface, which is represented on a display of the dentist's treatment station, at least one second input element for the generation of control information with which functions of the apparatuses are selectable and/or activatable independently of the navigation information generated by means of the first input element, and transfer means for the wireless transfer of the navigation and control information generated with the aid of the first and second input elements to the apparatuses or to a functional unit connected upstream of the apparatuses. The input device is so configured that the control information generated via the second input element is usable independently of a unit, e.g. a central server, administering the user interface, for control of the apparatus.

The present invention relates to an input device for the activation and control of functions of at least one apparatus at a dentist's or a dental treatment station/work station and to a dentist's or dental treatment station/work station having a plurality of apparatuses.

Modern dentist's or dental treatment stations or work stations have a plurality of different apparatuses, which are put to use in different working or treatment procedures. Merely as example, there is mentioned at this point dentist's treatment instruments such as drills or the like, optical examination apparatuses, imaging units such as displays, cleaning and care apparatuses for the treatment instruments or the patient chair. In order to bring together the various functions and tasks of these apparatuses in a common work station, the apparatuses are often connected with a central control unit, for example a central server, and exchange data therewith. The data and information arriving at the server can then, for example, be employed to coordinate the operational procedure of the individual apparatuses, or to protocol or log their use.

Often, beyond this, there is also the possibility of controlling or monitoring certain selected functions of the various apparatuses from the central server. On a display associated with the server, or provided at the work station, various user interfaces can be represented for this purpose, in which the functions made available from the respective apparatuses are represented, and, if applicable, can be selected. For example, through this it is made possible, with the aid of a screen menu, to alter the position of a patient chair or to set the speed of revolution or the torque of a dentist's handpiece from a central location. Further, the server can also be employed to control examination apparatuses, such as e.g. X-ray apparatuses or the like, or also merely serve to display information for monitoring the work station concerning the current activity of the apparatuses.

The activation and control of functions of the various apparatuses from a central point thus provides certain advantages with regard to the handling of the work station; however, often the server provided for the input of the various control commands is positioned, from an ergonomic point of view, at an unfavourable location within a dentist's practice. For example, if a dentist now wishes to carry out a treatment procedure, the possibility of central control of the various apparatuses, from the server, is not particularly helpful for him, since the server itself is as a rule not in the vicinity of the patient chair but at a certain distance there from, possibly being arranged even in a cupboard or another room. The aid provided by means of the server, and the possibilities for central control of the various apparatuses, in this case can thus not be used at all.

From U.S. Pat. No. 5,724,106 there is known a remote control device for the control of multimedia systems, which on the one hand has a trackball and on the other hand a plurality of selection keys, via which certain functions of the system can be purposively activated. All signals generated by means of the remote control are first transmitted to a central PC of the system, which then controls the various components of the multimedia system in appropriate manner.

The object of the present invention is first to indicate a possibility to activate, control and/or monitor in simple and comfortable manner, from a central location, the various functions of a plurality of apparatuses of a dentist's or dental treatment/work station. In particular the possibility is to opened up of being able to use the central control or monitoring even when the dentist is or must be at a certain location of the work station.

This object is achieved by means of the invention defined in the independent claims.

A first aspect of the present invention relates to an input device for the activation and control of functions of at least one apparatus of a dentist's or dental treatment/work station, wherein the input device has

a) a first input element for the generation of navigation information for the control of a pointer on a user interface, which is represented on a display of the dentist's treatment station,

b) at least a second input element for the generation of control information, with which, independently of the navigation information generated by means of the first input element, functions of the at least one apparatus are activatable,

c) and transfer means for wireless transfer of the navigation and control information generated with the aid of the first and second input elements to the apparatuses or a functional unit upstream of the apparatuses. In accordance with the invention the input apparatus is so configured that the control information generated via the second input element can be put to use independently of a unit, e.g. a central server, administering the user interface, for control of the apparatus.

In accordance with this first aspect of the invention there is thus provided a remote control with the aid of which the various functions of the different apparatuses can be activated, controlled and/or monitored. Through the employment of the remote control in accordance with the invention a user of the dental work or treatment station need no longer seek out the server directly in order to be able to use the central control functions. Instead, he can now undertake the control of the various apparatuses from any desired location, in particular also from the treatment chair.

Thereby, the input device in accordance with the invention has two input elements to be distinguished from one another.

A first input element is provided for the generation of navigation information which is employed for the control of a pointer on a user interface, which is represented on a display of the treatment/work station. As explained in the introduction, the various functions of the apparatus can often be called up, represented, and if applicable set, within the scope of certain user interfaces, wherein the user interfaces are differently configured, depending on the kind on the apparatus to be controlled. The first input element, the function of which is comparable with that of a normal computer mouse, takes this into account and is now employed for the purpose of carrying out navigation within these user interfaces. Preferably the first input element may be of a navigation element for the generation of navigation information and additional selection keys for the generation of supplementary selection information. In comparison to a conventional computer mouse, the navigation element thus assumes the function of the movement sensor of the mouse, whilst in contrast the function of the selection keys corresponds to the functions of the right and left mouse keys.

The input device of the present invention differs from a conventional wireless mouse in the making available of a second input element, which is provided for the generation of control information, with which certain functions of the apparatuses can be activated, independently of the navigation information generated by means of the first input element. Independently of the settings of the user interface currently represented on the display, with the aid of the further information element certain functions of the apparatus can thus be purposively called up. In this way there is the possibility of calling up individual, often used, functions of the apparatuses in a quick and uncomplicated manner—in particular without having to use the detour via the user interface. This direct control possibility, not known from U.S. Pat. No. 5,724,106, is also manifest in that the control information generated with the aid of the second input element is directly transferred to the apparatus concerned or to a so-called functional unit, and there put into action. The second input element can thus in particular be used independently of a server administering the user interface.

The second input element preferably comprises a function key field having a plurality of function keys, with which the individual desired functions of the apparatuses can be activated. Preferably at least a part of the function keys are provided for the purpose of selecting an image source desired for representation on a display of the work station, whilst in contrast with other keys the type of the representation can be influenced. Of course, the function keys can however also be associated with other functions.

Seen overall, the input device of the present invention opens up the possibility of calling up and activating, and if applicable setting, various functions of a dentist's or dental treatment or work station, comfortably from a central location.

A second aspect of the present invention relates to the transfer of the navigation and control information generated by the input device to the apparatuses. Although the possibility would be available of transferring at least the control information in each case directly to the various apparatuses, in order however to better exploit the possibilities of central control the transfer of the information to an intermediate functional unit is preferred, which in turn in reaction to the information received from the input device, controls the individual apparatuses. Thereby, the functional unit can in particular also be connected with the central server, in order to make possible the additional control of the apparatuses within the scope of the various user interfaces.

In accordance with a particularly preferred exemplary embodiment of the invention, it is provided that the functional unit passes on the navigation and control information received from the input device at least in part in a wireless manner to the further apparatuses of the treatment or work station. The transfer of the various information to the individual apparatuses is thus effected overall in a wireless manner, however, if applicable, with the diversion via the upstream functional unit connected with the server. Through this, the problem is simultaneously avoided that with a new arrangement of the individual apparatuses of the work station the laying of new cables would be necessary. Instead, the apparatuses can resume working directly after the new positioning.

In order to make possible disruption free data traffic between the various apparatuses it is preferably provided that the functional unit represents a master module within the scope of the wireless traffic, whilst in contrast the further apparatuses have or constitute the associated slave modules. A data transfer from the apparatuses to the functional unit and therewith to the server, is thus effected preferably first after a request on the part of the functional unit, so that the case cannot occur that a plurality of apparatuses transfer data at the same time. In this case, also the input device represents a slave unit, which however, is contacted by the master module at short intervals of time, in order to receive possible control and navigation information. Further, after assumption of contact between the functional unit and one of the apparatuses, also the possibility of a bi-directional data transfer would be conceivable.

A further aspect of the present invention relates to the representation of a variety of information on a display of the dentist's work or treatment station. Such displays are now widely used and can be employed for the representation of the most varied information. Thus, a first possibility for employment of this display consists in representing the user interface already mentioned above, with which the functions of the individual apparatuses can be controlled and activated. Beyond this, however, this display can also be employed to represent image information in the form of X-ray images, images of an intra-oral camera or other video information. This means that the most various kinds of image information are made available, which can be selectively represented on the display. Here, however, it is to be taken into account that the various image information is based in part on quite different formats, whereby in each case provision must be made for a suitable control of the display. Beyond this, at a modern dentist's work station, often even a plurality of displays are available on which in each case different information can or should be displayed.

In an advantageous further development of the present invention, there is thus provided an interface unit, via which, from a plurality of input signals containing image information, a desired signal can be selected and can be delivered as output signal to the display or in general to a screen. For this purpose the interface unit has
at least two inputs for receiving input signals containing image information
at least two outputs for transferring output signals, containing image information, to one or more displays and/or other interface units, connectable with the interface unit, and

at least one internal transfer unit for the selective passing on of the input signals containing image information to the outputs.

From the plurality of input signals made available there can thus be selected and provided for display the output signal which contains the desired image information. This function is achieved by means of a first transfer unit, designated in the following as a video matrix, which switches a plurality of different input video signals in any desired manner and independently of one another to different video outputs. Since at least some of the video signals as a rule are also combined with audio signals, there is preferably provided a further so-called audio transfer unit or audio matrix which in the same manner as in the video matrix switches the audio input signals to the corresponding outputs. Supplementary thereto there can finally also be put to use a second transfer unit as so-called VGA matrix, with which different VGA input signals—or general image signals corresponding to a PC—standard—can be switched independently to different VGA outputs. This VGA matrix can be employed for representing on the display or displays of a work station, image information obtained from various computers or servers.

The interface unit in accordance with the invention provides advantages in particular when a networking of various rooms of a practice is carried out. For example, there would now be the possibility of employing a central media server—for example a PC, a DVD player or a VHS player—the output signal which is transferred to a first work station having a first display and from there to further rooms of a practice. At each work station there can then be represented a desired video item.

A further module preferably provided in the interface unit is a digital processing unit with the aid of which image signals can be processed and issued in a modified form. This digital processing unit may for example be provided for the purpose of producing still images from certain video items, which are then represented on the or a further supplementary display. Also the simultaneous representation of a plurality of images, also of a single screen, is made possible through this. Further, by means of the processing unit in particular also a live conversion of analog video images to VGA data is made possible, so that these images can now also be directly represented, i.e. without the intermediary of a PC, on a monitor.

The interface unit likewise represents, in the sense of the present invention, an apparatus of the dentist's or dental treatment station/work station, which can be controlled in a wireless manner via the input device in accordance with the invention described above. Preferably the interface unit is integrated in the above-described functional unit, whereby the function keys provided in the second input element can be employed for the purpose of purposively bringing certain image information to representation on a display.

Below, the invention is to be described in more detail with reference to the accompanying drawings. There is shown:

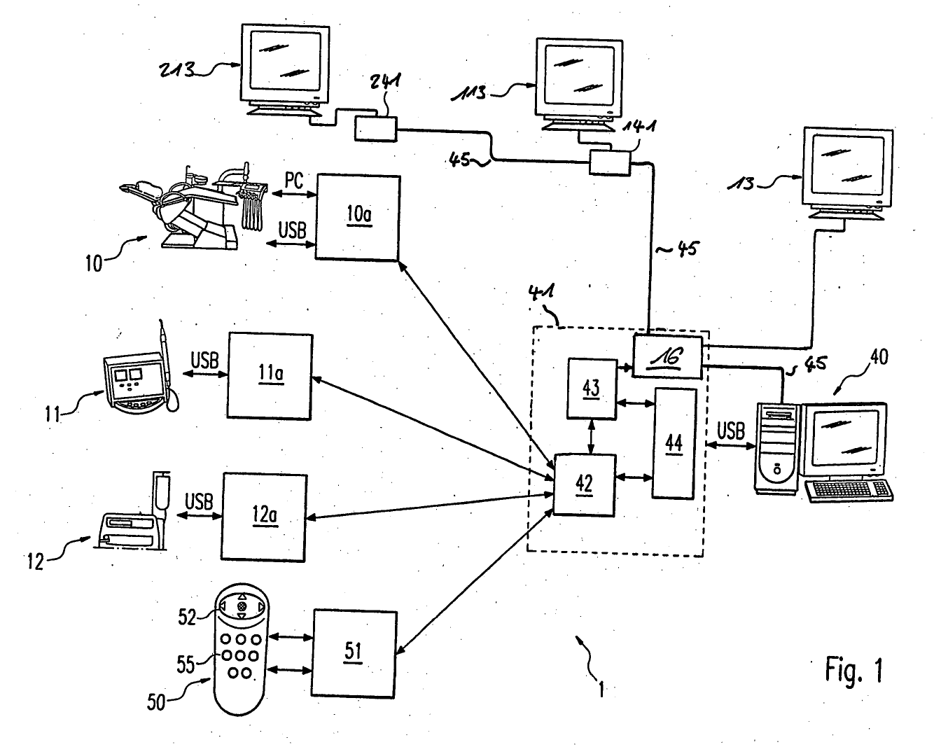

FIG. 1 schematically, the concept of the wireless central control of various apparatuses of a dentist's or dental treatment/work station;

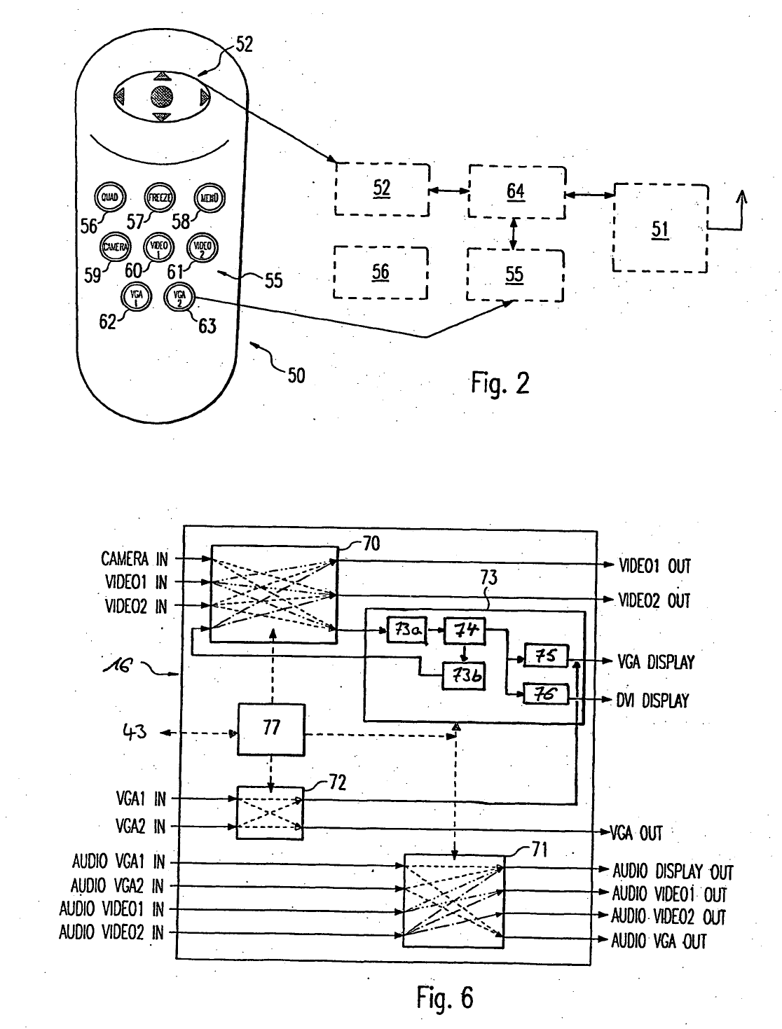

FIG. 2 an exemplary embodiment of an input device in accordance with the invention for the generation of navigation and control information;

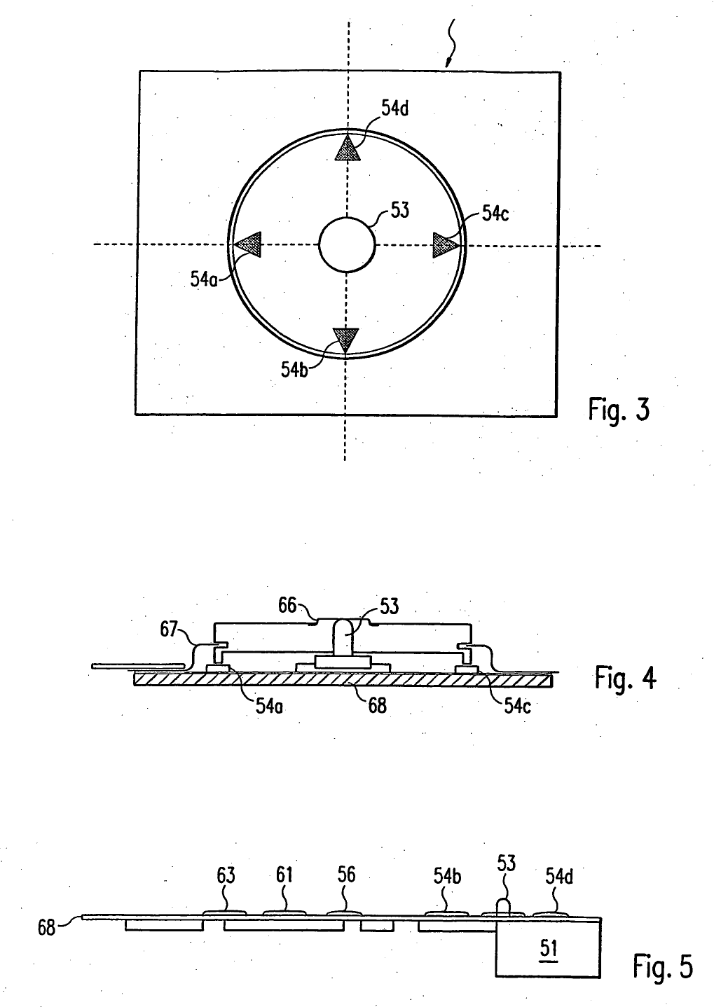

FIG. 3 an enlarged illustration of the first input element of the input device in accordance with the invention, for the generation of navigation information;

FIG. 4 the first input element, illustrated in FIG. 3, in a side section;

FIG. 5 the arrangement of various components for the realization of the input device illustrated in FIG. 2;

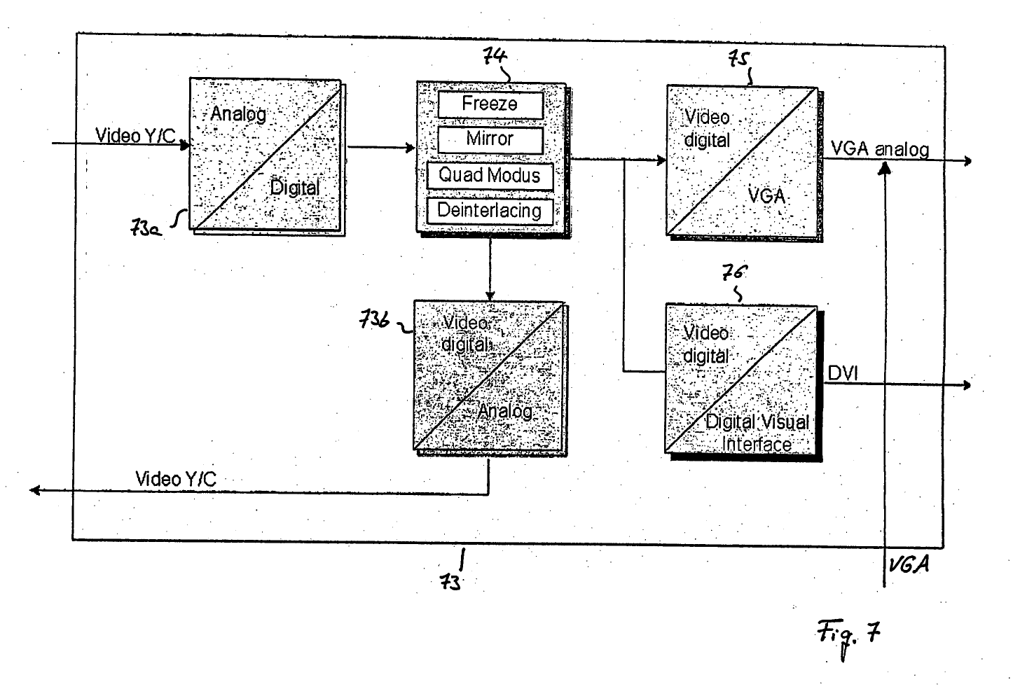

FIG. 6 a block circuit diagram of an interface unit in accordance with the invention; and

FIG. 7 an illustration to an enlarged scale of a central element of the interface unit.

The dentist's or dental treatment station or work station illustrated in FIG. 1, and designated in its entirety with the reference sign 1, has first a series of differing apparatuses which find employment in the activities of a dentist or dental technician. By way of example there are illustrated a dental treatment chair 10 with the associated treatment apparatuses, a laser diagnosis apparatus 11 and a dental motor handpiece 12. There belongs to these apparatuses, in the sense of the present application, moreover also an interface unit 16 associated with a display 13, which interface unit is a component of a so-called functional unit 41 and the task of which is to deliver to the display 13 suitable image and, if applicable, audio information. The function of this interface unit 16 will be described in more detail below.

The work of the various apparatuses is primarily coordinated or monitored by means of a central server 40, which is arranged usually at a certain distance from the treatment chair 10, for example in a cupboard or even in a special room of the practice. The control of the various apparatuses is thereby effected at least in part with the aid of the functional unit 41, which in a wireless manner transfers control commands to the various apparatuses, or receives data from these apparatuses. The more precise functioning of this functional unit 41 will be described in more detail below. It is further connected both with the server 40 and also with further functional units 113, 213 (if applicable, arranged in other rooms of the practice) via additional data or video lines 45, via which the signals containing video and image information can be transferred. Through this a kind of a practice-internal network is realized, with the aid of which image information made available from various sources can be selectively represented on the displays 13, 113, 213 associated with the three functional units 41, 141, 241.

The control and monitoring of certain functions of the apparatuses of the dentist's or dental treatment/work station is effected in two different manners.

First, it is possible to directly call up and, if applicable, activate certain selected functions of individual apparatuses. For example, by means of a single command, the optical diagnosis apparatus 11 could be started, the treatment chair 10 brought to a certain initial position, or a certain image signal delivered to the display 13. Other functions of the apparatus require, however, the additional indication of certain parameters, in order to be able to activate these functions in a desired manner. Thus, for example, a simple switching on of a dental X-ray apparatus would not be sufficient, since beyond this also information with regard to the required dose or the desired exposure time must be transferred.

In order to be able to call up also these more complex functions from a central location, the control of the apparatuses is effected in this case with the aid of a user interface represented on a display. For this purpose there is represented on the screen 13 or the display of the server 40 for example a menu, in which the various functions of the apparatus to be controlled can be selected and set. Here a pointer is then navigated over the user interface in order to select certain switch areas and to input desired parameters.

If the server 40 is at a different location than the various apparatuses of the work station, the central control of the apparatuses via the server is rather inconvenient, since the call up of a new function or the setting of a new parameter would in each case involve seeking out the server 40. In this case, the individual operation and decentral control of the apparatuses would be significantly more convenient.

In accordance with a first aspect of the present invention, the central control of the various apparatuses is however facilitated by means of the employment of an input device 50, with the aid of which control information can be input and transferred to the various apparatuses in a wireless manner. The input device 50, the configuration of which will be described in more detail below, stands in contact with the functional unit 41 via a transmitter and receiver unit 51, which receives information transferred from the input device 50 and in appropriate manner passes this on as control commands to the apparatuses of the treatment or work station or to the server 40. The functional unit 41 thereby stands in contact with the server 40 via a USB interface, in order to fully make use of the possibilities of central control of the various apparatuses.

Components of the functional unit 41 are first a transmitter and receiver unit 42, a controller 43, a so-called USB hub 44, establishing the connection to the server 40, and the above-mentioned interface unit 16 for the control of the associated display 13. The connection between the functional unit 41 and the server 40 is in particular necessary since a quantity of the information received from the operating unit 50, in particular when the information concerns the control or monitoring of an apparatus within the framework of a user interface represented on a display, must first be processed by means of the server 40 before it is passed on, if appropriate as corresponding control commands, to the apparatuses. For this purpose there serves the internal controller 43 of the functional unit 41, which passes on the information received from the server 40 to the transmission and reception module 42. The interface unit 16 is also controlled by the controller 43. Thereby it is to be taken into account that, with the described exemplary embodiment of the present invention, also control of the apparatuses 10, 11, 12 is effected via the server 40, whereby there is transferred to these apparatuses 10, 11, 12 control commands issued by the server 40 by means of the functional unit 41, which commands are appropriately acted upon by the apparatuses 10, 11, 12. A simplified version would, however, also be conceivable of employing the server 40 solely for the monitoring of the apparatuses 10, 11, 12 and for documentation. The apparatuses 10, 11, 12 would then receive no control commands from the server 40 but solely for their part transfer information via the functional unit 41 to the server 40, which would then be represented on the display. This information could relate both to the current operating condition of the apparatuses 10, 11, 12 and also to measurement results, e.g. the image data detected by an intra-oral camera. At least a partial control of the apparatuses 10, 11, 12 could, in this case, still be effected via the selection keys of the input device 50, the control information of which—as explained above, is passed on independently of the server 40.

The control or communication of the various apparatuses with the functional unit 41 is in turn preferably effected in a wireless manner. The control commands transferred from the transmission and reception unit 42 are thereby received by the transmission and reception units associated with the respective apparatuses (explicitly represented in FIG. 1 are the transmission and reception units 10a, 11a and 12a of the treatment chair 10, of the optical diagnosis apparatuses 11 and of the motor handpiece 12) and delivered to the apparatuses in digital form. Usually, the individual apparatuses are thereby connected with the corresponding transmission and reception units via USB interfaces, alternatively or additionally thereto there can however also be employed a conventional PC interface, as is illustrated in the case of the treatment chair 10.

Before the configuration of the operating apparatus 50 is described in more detail, it should be mentioned that the data communication between the functional unit 41 and the different transmission and reception units of the apparatuses is effected in accordance with the master-slave principle. Here, the transmission and reception unit 42 of the functional unit 41 represents the master module, which of its own account can transfer at any time signals to the other units, which constitute the slave modules. For their part, the slave modules transfer information, however, only after a request from the master module. In this manner an disruption-free data traffic between the individual units is ensured. Alongside this, however, in certain cases, the start of a data transmission can also be initiated from a slave unit. Further, it can be provided that after the log-in of an apparatus a bi-directional data transfer between the functional unit and the apparatus is permitted.

Although it would also be conceivable to connect the various apparatuses of the work station via data lines with the functional unit 41 or the server 40, the illustrated wireless communication between the functional unit 41 and the various apparatuses however brings certain advantages with it. Thus, on the one hand, through this obstacles for a person moving in the room are avoided. Beyond this, the apparatuses can be newly arranged in simple manner, without laying anew of communication lines being necessary.

In the following, the precise construction of the input device 50 is to be explained. As has already been mentioned, in the case of central control of the various apparatuses, it is necessary to distinguish between such functions which can be selected or monitored via a user interface represented on a display, that is such functions for which a navigation through the user interface is necessary, and such functions which can be directly called up and activated. In order to take account of these different requirements, the operating device 50 in accordance with the invention has two different input elements, namely a first input element 52 for the generation of navigation information for the control of a pointer on a user interface, and a further input element 55 for the generation of control information with which certain functions of the apparatuses can be directly called up and activated independently of the navigation information generated by the first input element.

It can be understood from the enlarged illustrations in FIGS. 2 and 3, that the first input element has a navigation element 53 for the generation of two-dimensional navigation information, and additional selection keys 54a to 54d for the generation of supplementary selection information. The navigation element is, in particular, a joystick 53, which is activated to initiate a synchronous movement of a pointer on the user interface. If a user holds the input device 50 in the hand, he can tilt the joystick 53 with the aid of his thumb in simple manner in the desired direction, in order to navigate the pointer in accordance with the selected direction over the user interface. In this sense, the joystick 53 assumes the function of a conventional computer mouse. In order to further complete this “simulation” of a computer mouse, the supplementary selection keys 54a to 54d are provided, which are arranged to the four sides of the joystick 53. By means of pressing down of the input element 52 in the region of one of the selection keys 54a to 54d, supplementary selection commands can be generated and transferred. In particular thereby, the functions of the right and left selection keys 54a and 54c correspond to the functions of the right and left mouse keys. The first input element 52 thus offers all functions which arise in similar manner with a conventional computer mouse.

In the illustrated preferred exemplary embodiment, beyond this, the joystick 53 has also a switching function in the third dimension, i.e. apart from tilting it can also be pressed down. Through this, the possibility is provided, in supplement to the four selection keys 54a to 54d, of generating a further fifth selection command.

The information generated during a so-called navigation mode, within the scope of the actuation of the first input element, is then made available with the aid of a serial RS232 or UART interface and converted by a microcontroller 64 into a transmissible protocol, which is issued via the transmission and reception unit 53 and transferred to the functional unit 41. The functional unit 41 in turn communicates via a USB interface with the server 40, which finally translates the navigation information into a movement of the pointer over the user interface represented on the display.

The user interface itself can for example be represented on the display 13 which is associated with the functional unit. The image information necessary for this may be generated in the server 40, but can be passed on via the lines 45 to any desired display, for which purpose in particular the interface units in the functional units 41, 141, 241 provide contributions. The doctor located at the treatment chair 10 then has the possibility of carrying out the central control of the individual apparatuses, made available by the server 40, directly from his current work station. In particular he need not actuate an input apparatus—e.g. the computer mouse connected with the server—located at the server, in order to navigate through the various menu points of the user interface.

FIG. 4 shows an exemplary embodiment for the realization of the first input element 52. The joystick and the various selection keys 54a to 54d are thereby arranged on a board 68 and held via rubber bearings 62 and 67. Upon a pressing down or tilting of the rubber bearings into a desired position, the corresponding switches are activated, through which the desired selection information is generated.

In contrast to the first input element 52, which is responsible for the generation of navigation information, the second input element 55 serves—as explained above —for the purpose of activating purposively certain functions of the individual apparatuses. The second input element 55 is, for this purpose, constituted as a function key field, which in the illustrated exemplary embodiment has overall eight function keys 56 to 63. The information generated upon a pressing down of these function keys is likewise delivered to the microcontroller 64, by a further interface circuit 55, which microcontroller in turn translates this information into a transmissible protocol and transfers it to the transmission and reception unit 51. Beyond this, the input device 50 has also a battery supply 56, which makes possible a wireless operation of the apparatus.

FIG. 5 again shows, in side section, a possibility for the realization of the input device in accordance with the invention. The various elements are thereby in the form of a joystick 53 and a plurality of foil keys arranged on the board 68, on the underside of which the electrical components and the transmission and reception unit 51 are located.

Below, the functions of the individual function keys 56 to 63 of the second input element 55 are now to be explained. It is self-evident that here a specific exemplary embodiment is involved, in which the function keys 56 to 63 are provided primarily for the control of the interface unit 16 located in the functional unit 41. Of course, the keys could however also be provided with other functions for the control of one of the apparatuses 10, 11 or 12.

The three upper keys 56-58 serve in the illustrated example for the control of the image representation on the display 13 associated with the interface unit 16. With the aid of the first key 56 (“QUAD”) the representation on the display 13 can initially be switched into a so-called QUAD representation, in which four different video images are represented on the screen at the same time.

In particular there is opened up through this the possibility of combining the representation of a live image with the simultaneous display of three still images or e.g. to represent four still images at the same time.

The second key 57 (“FREEZE”) serves to freeze the image currently transferred from the selected image source, that is to generate a still image on the display.

The right key 58 finally serves exclusively to change from the above-described navigation mode into a so-called menu mode, in which the further navigation through the user interface represented on the display 13 is effected not on the basis of two-dimensional displacement of a pointer but by means of the change between various menu points. This menu mode also has effect on the functioning of the first input element 52, since hereby only the four selection keys 54a to 54d are active and serve the purpose of changing to a right, left, upper or lower menu point. The joystick 53 itself is inactive during the menu mode and the serial RS232 interface is blocked.

The function keys 59 to 63 of the two lower rows of the key field 54 serve exclusively for the selection of an image source, the information of which is to be represented on the display 13. Via these keys it is thus determined which image signal of the work station should passed onto the display 13. With the aid of the three upper keys 59, 60 and 61, there can thereby be switched between the intra-oral camera and two further video input signals, whereas in contrast the two upper keys 62 and 63 are provided for the selection of a VGA input signal. With the aid of these two upper keys it can for example be selected from which computer—for example from the server 40 or a computer (not shown) associated with a treatment chair 10—image information should be transferred and represented on the display 13.

As has already been mentioned, there is of course also the possibility of providing the function keys of the key field 55 with other functions. For example, these keys can be employed to activate certain basis settings of various apparatuses—of the patient chair 10 or of the optical diagnosis apparatus. Likewise it would be conceivable to assign to all or at least some of the keys respective changeable functions, which are newly defined depending on the kind of the momentarily selected menu point on the user interface. It is of significance however that the navigation information generated with the aid of the first input element 52 is passed onto the server 40, in order through this to make possible a navigation on the screen interface and therewith, if applicable, an indirect control of the apparatuses 10, 11, 12, whereas in contrast the information generated upon actuation of the individual function keys 52 to 63 brings about a direct control of the corresponding apparatus. In the illustrated exemplary embodiment, e.g. the functional unit 41 or the interface unit 16 located therein is directly, and without the intermediary of the server 40, controlled.

Overall, the operating device 50 in accordance with the invention opens up the possibility of controlling the various apparatuses of a treatment station in a convenient manner, without for this in each case having to seek out a specific input device located at the server 40 responsible for the central control.

From the above explanation, it is apparent that at a dentist's work and treatment station the most varied image information is available, which can be represented on a display. This image information can be made available from the most varied sources. One conceivable source would be, for example, an intra-oral camera, the images of which are represented on a display during the examination, or an examination apparatus, e.g. a dental x-ray apparatus, the examination results of which can likewise be shown on the display in the form of x-ray images. A PC—e.g. the server 40—of course also represents a further image source, via which information regarding the patient or operating parameters of an examination or treatment apparatus currently being used are represented.

Since the association of the different image information to the various displays within the treatment room or the overall practice is, with the employment in each case of separate lines and cables, complex and liable to faults, there will be described below a possibility of associating the various image information with individually determined displays. This task is fulfilled by means an interface unit 16 connected upstream of the displays 13, 113 and 213, which interface unit is to described below in more detail with reference to FIGS. 6 and 7.

The task of the interface unit 16 is, as mentioned, to select a desired signal from the plurality of image information items available and to deliver this to the associated display as output signal. For this purpose the interface unit 16 has overall four internal units, a first transfer unit (the so-called video matrix 70), an audio transfer unit or audio matrix 71, a second transfer unit (the so-called VGA matrix 72) and a digital processing unit 73. All four units are controlled from a central controller 77, which co-ordinates the functions of the four units amongst one another. The controller itself in controlled by the controller 43 of the functional unit 41 in which, in the illustrated exemplary embodiment, the interface unit 16 is integrated. In an alternative form of embodiment, the interface unit 16 could however also represent a separate apparatus, which just as the further apparatuses 10, 11, 12 of the dentist's or dental treatment or work station can be controlled via the input device 50 in a wireless manner and, if applicable, via the server 40 and functional unit 41.

The task of the video matrix 70 is to switch four different input signals, in any desired manner, to three outputs. Input signals are delivered to the video matrix 70 via three external terminals (CAMERA IN, VIDEO1 IN, VIDEO2 IN) and via an internal terminal connected with the processing unit 73. In accordance with the commands of the controller 77, the video matrix 70 then associates the corresponding input signal with three outputs, whereby two of the outputs (VIDEO 1 OUT and VIDEO2 OUT) can be connected directly with a representation unit—for example a video screen. These outputs can, however, also be employed for passing on the video signal to a further interface unit or to the functional units 141, 241 having interface units located therein, through which a networking of the interface units of a dental practice amongst one another is attained. The third output of the video matrix 70 finally leads to the digital processing unit 73, which is employed for processing the video image.

Internally, the processing unit 73 has, corresponding to the illustration in FIG. 7, for this purpose first a translation block 73a, which transforms the analog video signal into a digital signal. The digital signal can then be selectively processed in a processing block 74, whereby corresponding to the representation the possibilities “Freeze”, “Mirror”, “Quad” and “Deinterlacing” are available. The video signal processed in this manner may be then either—after preceding conversion into an analog signal by means of a further conversion block 73b—again be delivered to the video matrix 70 and via this delivered to one of the two video outputs or to further conversion blocks 75 and 76. These further conversion blocks 75 or 76 are responsible for forming from the digital video signal a signal corresponding to the VGA standard or the DVI (Digital Video Interface) standard. The VGA signal can then be delivered to an output (VGA DISPLAY) which is for example connected with a computer monitor, whereas in contrast the DVI signal can be delivered to a further output (DVI DISPLAY). Significant here is that the processing unit 73 opens up in particular also the possibility of converting the input side analog video data “live” into signals corresponding to the VGA standard, so that without the intermediary of a further apparatus an immediate representation also of moving images is made possible on a monitor.

Supplementary to the processing of the video input signals, the interface unit 16 may also process two further VGA input signals via the two inputs VGA1 IN and VGA2 IN. These two inputs are connected with the VGA matrix 72 to which are delivered the signals selectively either of the processing unit 73 for passing on to a VGA display or a further output (VGA OUT) or for passing onto a further unit—for example the server or the like.

It is to be remarked that the mentioned VGA standard is noted merely by way of example. Of course, the processing unit 73 and the two conversion units 72 can also be provided for the purpose of processing or generating signals corresponding to another PC graphic standard, whereby however also for other graphic standards the possibility of “live” conversion of analog video images is available.

Often, the video or VGA input signals are combined with audio information. In order to take this into account upon the passing on of the various image providing signals, the audio matrix 71 is provided for the purpose of diverting the audio signals associated with the two input signals and the two VGA input signals in a desired manner also to the corresponding video and VGA output terminals. In this manner it is ensured that an audio signal associated with an input image signal is also switched to the corresponding associated output lines.

The employment of this interface unit 16 now opens up the possibility of connecting various displays, which are arranged in one or different practice rooms, with one another via a network. For example, in a central location a media sever can be established, which is a PC or a video apparatus such as e.g. a DVD or a video player, the output signals (video and audio) of which are transferred to a first work station and from there to the further praxis rooms, more precisely to the further interface units located in these practice rooms. Through this it can be ensured that at each work station the same video item is shown. This possibility of networking is made possible, as illustrated in FIG. 1, by means of the connection of a plurality of functional units with the interface units arranged therein. Thereby, for example, the functional units 141, 241, with the displays 113, 213 associated therewith, may be arranged in other rooms of the practice, whereby via the lines 45 the network for the selective passing on of the image and audio information is constituted.

A further exemplary embodiment consists in forming a still image from a signal delivered from the intra-oral camera and passing on this still image to one or more work stations via the interface unit(s). In particular, different image signals can be selectively directed to different displays. Of significance here is that the different outputs of the interface unit in accordance with the invention can be occupied selectively with the desired image signals, whereby, if applicable, the associated audio information can be passed on in the said manner.

By means of the interface unit in accordance with the invention the passing on of video and audio signals is thus significantly facilitated. In particular, it is no longer necessary in each case to lay a plurality of lines from one image producing unit to the various display or representation units.

Thus, overall, the present invention opens up the possibility of realizing a dentist's or dental work station or treatment station which is comfortable to operate. Thus, a central control of the various apparatuses is made possible with the aid of a remote control, by means of which the advantages of networking of the apparatuses with one another can be effectively exploited. At the same time the most varied image-representing data can be coupled out in desired manner or delivered to determined display or representation units, so that diverse possibilities for the representation of image representing information are attained.

1. Input device for the activation and control of functions at least one apparatus of a dentist's or dental treatment station/work station, the input device comprising:
a first input element for the generation of navigation information for the control of a pointer on a user interface, which is represented on a display of the dentist's treatment station,
at least a second input element for the generation of control information, with which, independently of the navigation information generated by the first input element, functions of the apparatuses are selectable and/or activatable,
a transfer device for the wireless transfer of the navigation and control information generated with the aid of the first and second input elements to the apparatuses or to a functional unit connected upstream of the apparatuses,
wherein the control information generated via the second input element is useable for control of at least one apparatus independently of the unit administering the user interface.

2. Input device according to claim 1,
wherein
the first input element comprises a navigation element for the generation of two-dimensional navigation information, and at least two selection keys for the generation of supplementary selection information.

3. Input device according to claim 2,
wherein
the navigation element is a joystick.

4. Input device according to claim 3,
wherein
for the generation of an additional item of selection information, the joystick can be pressed down.

5. Input device according to claim 2,
comprising a common interface for passing on
the information generated by the navigation element and the selection keys in a navigation mode.

6. Input device according to claim 5,
wherein
in the navigation mode, the common interface is a UART interface.

7. Input device according to claim 2,
wherein
in a menu mode, the function of the navigation element is blocked, and only selection information can be generated with the aid of the selection keys.

8. Input device according to claim 7,
wherein
the first input element comprises two additional selection keys for the generation of further selection information in the menu mode.

9. Input device according to claim 1,
wherein
the second input element is formed by a function key field having a plurality of function keys.

10. Input device according to claim 9,
wherein
a part of the function keys is provided for the control of an interface unit for the selective transfer of video and/or audio signals.

11. Input device according to claim 9,
wherein
a part of the function keys is provided for the selection of a video source provided for representation on a display.

12. Input device according to claim 9,
wherein
a part of the function keys is provided for selection of an image signal, provided for representation on a display, corresponding to a PC graphic standard.

13. Dentist's or dental treatment station/work station, comprising
plural dental work apparatuses and/or examination apparatuses,
an input device for the generation and wireless transfer of navigation and/or control information for the activation and control of functions of the apparatuses and
a functional unit, connected upstream of the apparatuses, which receives the navigation and/or control information transferred from the input device and passes this onto the apparatuses.

14. Dentist's or dental treatment/work station according to claim 13,
wherein
the functional unit passes on the navigation and/or control information transferred from the input device to the apparatuses at least partially in a wireless manner.

15. Dentist's or dental treatment/work station according to claim 14,
wherein
the functional unit comprises a master module for wireless communication with the apparatuses, whereby in each case a slave module is associated with the apparatuses, which slave module passes on the information received from the master module to the associated apparatus.

16. Dentist's or dental treatment/work station according to claim 15,
wherein
the slave modules are integrated in the respective apparatuses or connected with these via a RS232 interface and/or a PC interface.

17. Dentist's or dental treatment/work station according to claim 13,
wherein
the input device can generate
navigation information for the control of a pointer on a user interface, which is represented on a display of the dentist's treatment station, and
control information with which functions of the apparatuses are selectable and/or activatable independently of the navigation information.

18. Dentist's or dental treatment/work station according to claim 17,
wherein
the input device comprising the activation and control of functions at least one apparatus of the treatment station/work station, the input device comprising:
a first input element for the generation of navigation information for the control of a pointer on a user interface, which is represented on a display of the dentist's treatment station,
at least a second input element for the generation of control information, with which, independently of the navigation information generated by the first input element, functions of the apparatuses are selectable and/or activatable, and
a transfer device for the wireless transfer of the navigation and control information generated with the aid of the first and second input elements to the apparatuses or to a functional unit connected upstream of the apparatuses,
wherein the control information generated via the second input element is useable for control of at least one apparatus independently of the unit administering the user interface.

19. Dentist's or dental treatment/work station according to claim 13,
wherein
the functional unit further stands in connection with a server, whereby a data exchange between the server and the apparatuses takes place via the functional unit.

20. Dentist's or dental treatment/work station according to claim 18,
wherein
the functional unit is connected with the server via a USB interface.

21. Dentist's or dental treatment/work station according to claim 13,
wherein
at least one of the apparatuses controlled by the functional unit is an interface unit comprising:
at least two inputs for receiving input signals containing image information,
at least two outputs for the passing on of output signals containing image information to one or more displays connectable with the interface unit and/or to further interface units, and
at least one internal transfer unit for selective passing on of the input signals containing image information to the outputs.

22. Dentist's or dental treatment/work station according to claim 21,
wherein
at least one input signal is a video signal.

23. Dentist's or dental treatment/work station according to claim 22,
wherein
the interface unit comprises at least two inputs and two outputs for video signals and a first transfer unit, via which the video input signals are selectively passed onto the outputs.

24. Dentist's or dental treatment/work station according to claim 23,
wherein
the interface unit has an internal processing unit for the transformation or processing of analog video signals, whereby the processing unit has on the input side a first transformation block for the transformation of the analog video signal into a digital signal.

25. Dentist's or dental treatment/work station according to claim 24,
wherein
the digital signal produced by the first transformation block is deliverable to a processing block for digital processing of the video signal.

26. Dentist's or dental treatment/work station according to claim 24,
wherein
the digital signal produced by the first transformation block and, if applicable, processed by the processing block, is selectively deliverable to the first transfer unit or to at least one further transformation unit for the generation of a signal corresponding to a PC graphic standard.

27. Dentist's or dental treatment/work station according to claim 26,
wherein
the second transformation unit produces a video signal corresponding to the VGA standard.

28. Dentist's or dental treatment/work station according to claim 27,
wherein
the digital signal produced by the first transformation block and, if applicable, processed by the processing block, is deliverable to a third transformation unit for the generation of an output signal corresponding to the DVI standard.

29. Dentist's or dental treatment/work station according to claim 21,
wherein
at least one input signal is a signal corresponding to a PC graphic standard.

30. Dentist's or dental treatment/work station according to claim 29,
wherein
the interface unit comprising at least two inputs and two outputs for signals corresponding to the PC graphic standard, and a second transfer unit via which the signals are selectively passed on to the outputs.

31. Dentist's or dental treatment/work station according to claim 29,
wherein
the signals corresponding to the PC graphic standard are VGA signals.

32. Dentist's or dental treatment/work station according to claim 21,
wherein
the interface unit comprises at least two inputs and outputs for audio signals, which in each case are associated with the inputs and outputs for signals containing image information, and an audio transfer unit via which the audio signals at the inputs are passed on corresponding to the passing on of the signals containing image information to the associated outputs.
